(19) United States
(12) Patent Application Publication
(10) Pub. No.: US 2025/0176838 A1
(43) Pub. Date: Jun. 5, 2025

(54) SYSTEMS, ASSEMBLIES, AND METHODS FOR ASSESSMENT AND MANAGEMENT OF CONGESTION IN HEART FAILURE

(71) Applicant: Endotronix, Inc., Naperville, IL (US)

(72) Inventors: Angad SINGH, Naperville, IL (US); Omid FOROUZAN, Chicago, IL (US)

(21) Appl. No.: 19/042,751

(22) Filed: Jan. 31, 2025

Related U.S. Application Data
(63) Continuation of application No. PCT/US2023/029410, filed on Aug. 3, 2023.
(60) Provisional application No. 63/394,764, filed on Aug. 3, 2022.

(51) Int. Cl. A61B 5/02; A61B 5/00; A61B 5/021; A61B 5/11

(52) U.S. Cl. CPC A61B 5/02028; A61B 5/02007; A61B 5/021; A61B 5/1116; A61B 5/6804; A61B 5/6876; A61B 2560/045; A61B 2562/0219

(57) ABSTRACT

Disclosed are systems and methods for assessment, management, and treatment of congestion in heart failure. The systems and methods may include or relate to wireless implants, reader modules, and uses thereof, which are configured to measure hemodynamic parameters of a patient in different patient states and positions. For example, the reader module may be hands free. For example, the reader module may be used at home by a patient. The reader module may be able to take readings and measurements when the patient is in a multitude of positions, including but not limited to, the supine and the prone positions. The reader module may be able to communicate and receive data from an implanted device when in a certain proximity of the implanted device and may be able to store, analyze, and/or transmit data to a different wireless device over a network, for example. In an embodiment, the reader module may be portable and wearable, e.g., through use of a garment or strap.

### CROSS REFERENCE TO RELATED APPLICATIONS

The application is a continuation of International Application No. PCT/US2023/029410 filed on Aug. 3, 2023, entitled “SYSTEMS, ASSEMBLIES, AND METHODS FOR ASSESSMENT AND MANAGEMENT OF CONGESTION IN HEART FAILURE, which claims priority to U.S. Patent Application No. 63/394,764, filed on Aug. 3, 2022, entitled “SYSTEMS, ASSEMBLIES, AND METHODS FOR ASSESSMENT AND MANAGEMENT OF CONGESTION IN HEART FAILURE,” each of which are incorporated herein by reference in their entireties.

### FIELD OF THE INVENTION

The present disclosure relates generally to systems, methods, and assemblies for assessment, management, and treatment of congestion in heart failure and, more particularly, to wireless implants, reader devices, and uses thereof, that are configured to measure hemodynamic parameters of a patient in different patient states and positions.

### BACKGROUND

Chronic heart failure, also known as congestive heart failure or “HF,” is a progressive medical condition where the heart is unable to pump blood efficiently. As a result, blood returns to the heart faster than it can be pumped out, and the heart can become congested. The back-up prevents oxygen-rich blood from reaching the body's other organs and extremities. Without intervention, chronic heart failure worsens over time and can contribute to morbidity.

Chronic heart failure presents a significant global health concern with an increasing prevalence worldwide due to various factors including aging populations, lifestyle changes, and the increasing incidence of risk factors such as obesity, hypertension, and diabetes. The increasing prevalence has lead to a rapid escalation of cardiovascular mortality and morbidity with a consequent health economic burden. The estimated medical costs associated with chronic heart failure are also substantial due to the need for ongoing medical care, hospitalizations, medications, and other treatments to manage the condition and its complications. It is estimated that the overall increase of chronic heart failure incidence, morbidity, and mortality will result in an increase of billions of dollars in total direct medical costs over the next several years.

Congestion is a common complication of chronic heart failure defined as the signs and symptoms of extracellular fluid accumulation which can lead to functional deterioration and cardiac decompensation that often result in patients going to the emergency room and/or hospital. One mechanism of congestion is the progressive accumulation of sodium and water (volume overload) which typically results in an increase in weight of the patient in the weeks prior to hospitalization. Another mechanism of congestion is changes to the capacitance of the veins (volume misdistribution or redistribution), in particular the splanchnic veins, that stores a large portion of the blood in circulation. Depending on the dominant underlying mechanism, treatment of congestion may vary. For example, congestion due to volume overload may be treated with diuretics whereas treatment of congestion due to volume misdistribution or redistribution may be treated with vasodilators.

Pulmonary artery pressures (“PAP”) can increase early in the stages of congestion, before clinical signs and symptoms of chronic heart failure develop. Therefore, monitoring of these pressures may provide a prompt and tailored treatment for patients.

Conventional methods for measuring PAP include right heart catheterization. During right heart catheterization, a thin, flexible catheter is inserted into a blood vessel, usually through the groin, neck, or chest, and advanced through the veins until it reaches the right atrium and right ventricle of the heart. From there, the catheter can be further advanced into the pulmonary artery to measure pressures in the pulmonary circulation and to assess cardiac function. The catheter can communicate pressure from its distal end to a transducer outside the body. Catheters with wired in-body microsensors may also be used. Right heart catheterization, however, can only be performed in the clinic and is not suitable for home monitoring.

Conventional, noninvasive methods for measuring using implantable sensors can be used for home monitoring, but are limited in use and functionality. For example, Abbott Medical's CardioMEMS system has been used to monitor PAP of patients at home, but the system requires a large, stationary external reader that plugs into a wall outlet and requires the patient to lie supine during measurements. The system is not amenable to measurements in different patient states and positions. In another example, the V-Lap device by Vectorious Medical and its reader are configured to measure Left Atrial Pressure (LAP), another hemodynamic parameter that can be a surrogate for PAP. While smaller than CardioMEMS and battery powered, the V-Lap reader requires the patient to circle the thoracic cavity with a large, sash-like antenna strap, any movement of which during the reading can cause unacceptable inaccuracy.

As a result, there is a need for improved systems, methods, and assemblies for assessment, management, and treatment of congestion in heart failure and, more particularly, to wireless implants, reader devices, and uses thereof, that are configured to measure hemodynamic parameters of a patient in different patient states and positions. More particularly, improved systems, methods, and assemblies are needed that allow fast and simple wireless communication with permanently implanted sensors to measure PAP and PAP waveform derived hemodynamic parameters such as stroke volume, cardiac output, pulmonary vascular resistance, total pulmonary resistance, vascular, and the like.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure. Any of the described aspects may be isolated or combined with other described aspects without limitation to the same effect as if they had been described separately and in every possible combination explicitly.

Disclosed are systems and methods for assessment, management, and treatment of congestion in heart failure. The systems and methods may include or relate to wireless implants, reader modules, and uses thereof, that are configured to measure hemodynamic parameters of a patient in different patient states and positions. For example, the reader module may be hands free. For example, the reader module may be used at home by a patient. The reader module may be able to take readings and measurements when the patient is in a multitude of positions, including but not limited to, the supine and the prone positions. The reader module may be able to communicate and receive data from an implanted device when in a certain proximity of the implanted device and may be able to store, analyze, and/or transmit data to a different wireless device over a network, for example. In an embodiment, the reader module may be portable and wearable, e.g., through use of a garment or strap.

Disclosed is a method for assessment and management of congestion in heart failure. In an embodiment, the method may comprise implanting a sensor into a circulatory system of a patient. In an embodiment, the method may comprise coupling an external reader module to the sensor. In an embodiment, the method may comprise recording at least one measurement from the sensor with the external reader module. In an embodiment, the external reader module may detect a position the patient is in when the at least one measurement is recorded. In an embodiment, the external reader module may correlate the at least one measurement with the position detected.

In an embodiment, the external reader module may include an accelerometer or tilt sensor. In an embodiment, the external reader module may be is configured to record measurements from the sensor when the patient is in any position. In an embodiment, the external reader module may be is configured to record measurements from the sensor when the patient is doing any of laying down, walking, exercising, standing, sitting, and sleeping. In an embodiment, the method may further include recording at least two measurements from the sensor with the external reader module when the patient is in at least two different positions. In an embodiment, the method may further include analyzing the at least one measurement against the position detected and making a determination, wherein the determination evaluates whether the at least one measurement is within a range associated with the position detected. In an embodiment, the analyzing and determination steps may occur in a cloud network. In an embodiment, the sensor may be implanted in a patient's pulmonary artery. In an embodiment, the at least one measurement may be a pulmonary artery pressure. In an embodiment, the method may be carried out with no other device than the sensor and external reader module. In an embodiment, the coupling and recording steps may occur at home or outside of a clinical setting.

Disclosed is a system for assessment and management of congestion in heart failure. In an embodiment, the system may comprise an implantable sensor configured to insert into a circulatory system of a patient. In an embodiment, the system may comprise an external reader module configured to selectively couple with the implantable sensor. In an embodiment, the external reader module may record measurements taken from the implantable sensor. In an embodiment, the external reader module may include an accelerometer configured to detect a posture and position of the patient. In an embodiment, the external reader module may be configured to record measurements when the patient is in any position.

In an embodiment, the external reader module may be configured to record measurements when the patient is in more than one position. In an embodiment, the external reader module may be configured to record measurements when the patient is in a supine and an upright position. In an embodiment, the external reader module may be configured to record measurements when the patient is laying down, walking, exercising, standing, sitting, and sleeping. In an embodiment, the external reader module may be hands-free. In an embodiment, the system may further include an adhesive configured to attach the external reader module to the patient. In an embodiment, the system may further include a garment configured to attach the external reader module to the patient.

In an embodiment, the external reader module may comprise a first module and a second module. In an embodiment, the first module may be configured to attach to the patient near the implantable sensor. In an embodiment, the second module may be configured to attach to the patient anywhere on the patient's body. In an embodiment, the first module may include only an antenna for near field or mid-field communication with the implantable sensor and an adhesive configured to attach first module to the patient. In an embodiment, the first module may be single use and disposable.

The following description and the drawings disclose various illustrative aspects. Some improvements and novel aspects may be expressly identified, while others may be apparent from the description and drawings.

### BRIEF DESCRIPTION OF THE DRAWINGS

The present teachings may be better understood by reference to the following detailed description taken in connection with the following illustrations, in which like reference characters refer to like parts throughout, wherein:

FIG. 1 shows an embodiment of an exemplary wireless sensor known in the art for implant and positioning in a patient's pulmonary artery to measure pressure;

FIG. 2 shows an embodiment of an exemplary wireless reader known in the art for coupled use with an implanted wireless sensor to receive pressure measurements;

FIG. 3 shows an embodiment of the wireless reader of FIG. 2 used by a patient and placed near an implanted wireless sensor, such as that shown in FIG. 1;

FIG. 4 shows an enlarged view of the wireless reader of FIG. 2 used by a patient and placed near the wireless sensor of FIG. 1 as implanted into the patient;

FIG. 5 shows an embodiment of an exemplary wireless sensor system known in the art;

FIG. 6 shows an embodiment of a reader module as positioned on a patient by a strap or garment and a user device in accordance with various disclosed aspects herein;

FIG. 7A shows an embodiment of a singular module of a reader module and a strap or garment in accordance with various disclosed aspects herein;

FIG. 7B shows an embodiment of split modules of a reader module in accordance with various disclosed aspects herein;

FIG. 8 shows a graphical representation of longitudinal data display of parameters obtained in seated and supine positions in accordance with various disclosed aspects herein;

FIG. 9 shows an embodiment of a data communication system including an implantable sensor, reader module, and user device in accordance with various disclosed aspects herein.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Reference will now be made in detail to exemplary embodiments of the present teachings, examples of which are illustrated in the accompanying drawings, wherein like numbered aspects refer to a common feature throughout. It is to be understood that other embodiments may be utilized and structural and functional changes may be made without departing from the respective scope of the present teachings. Moreover, features of the various embodiments may be combined or altered without departing from the scope of the present teachings. As such, the following description is presented by way of illustration only and should not limit in any way the various alternatives and modifications that may be made to the illustrated embodiments and still be within the spirit and scope of the present teachings.

In this disclosure, numerous specific details provide a thorough understanding of the subject disclosure. It should be understood that aspects of this disclosure may be practiced with other embodiments not necessarily including all aspects described herein, etc.

As used herein, the words “example” and “exemplary” means an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

Further, unless context suggest otherwise, descriptions of shapes (e.g., circular, rectangular, triangular, etc.) refer to shapes meeting the definition of such shapes and general representation of such shapes. For instance, a triangular shape or generally triangular shape may include a shape that has three sides and three vertices or a shape that generally represents a triangle, such as a shape having three major sides that may or may not have straight edges, triangular like shapes with rounded vertices, etc.

Disclosed are systems and methods for assessment, management, and treatment of congestion in heart failure. The systems and methods may include or relate to wireless implants, reader modules, and uses thereof, which are configured to measure hemodynamic parameters of a patient in different patient states and positions. For example, the reader module may be hands free. For example, the reader module may be used at home by a patient. The reader module may be able to take readings and measurements when the patient is in a multitude of positions, including but not limited to, the supine and the prone positions. The reader module may be able to communicate and receive data from an implanted device when in a certain proximity of the implanted device and may be able to store, analyze, and/or transmit data to a different wireless device over a network, for example. In an embodiment, the reader module may be portable and wearable, e.g., through use of a garment or strap.

As described, pulmonary artery pressures (“PAP”) can provide indication of the stages and extent of heart congestion and failure before clinical signs and symptoms of chronic heart failure develop. Therefore, monitoring of these pressures, and other related pressures including, but not limited to, right atrial pressure, right ventricular pressure, pulmonary capillary wedge pressure, cardiac output, and the like, may provide a prompt and tailored treatment for patients. Such monitoring and treatment may be able to mitigate and prevent escalation of heart failure. Although the disclosure may generally refer to pressure and pulmonary artery pressures, it is noted that any other hemodynamic parameters and pressures in different locations of the cardiovascular system may also be measured and analyzed in accordance with this disclosure.

Further, longitudinal monitoring of these pressures at home may be more accessible and convenient to patients and may provide an even more prompt and tailored treatment for patients with chronic heart failure, aiming to avoid functional deterioration, cardiac decompensation, and emergency visits and hospitalizations due to congestion. Also, measurement of PAP pressure and other hemodynamic parameters with the patient in different postures (i.e., under postural or orthostatic stress) may provide additional insight regarding the mechanisms of congestion at play and lead to more precise treatment. The described systems and methods can be used to provide both at home monitoring as well as monitoring in multiple patient positions, for example.

In an embodiment, the described systems and methods can provide small, highly portable, easily wearable reader devices, and systems and methods thereof, which can be operable quickly and efficiently by the patients themselves, in a variety of patient states and positions. The devices can be capable of stable, accurate measurement during different postures such as supine, seated, prone, and upright positions. In an embodiment, seated measurements may be useful for patients with heart failure they may have difficulty breathing when they have to lay on their back. As described herein, measurements taken in upright versus supine postures and positions, and comparing the readings, may be able to provide insight into the patient's heart or general health. In an embodiment, the device and system data may be coupled with treatment algorithms that can diagnose the mechanisms, stages, and characteristics of congestion in the patient and guide appropriate treatments and interventions. The reader devices and systems may include motion sensors and algorithms to detect and classify the posture to correlate the posture with the measured pulmonary artery pressure which may similarly be coupled with treatment algorithms that can diagnose the mechanisms, stages, and characteristics of congestion in the patient and guide appropriate treatments and interventions.

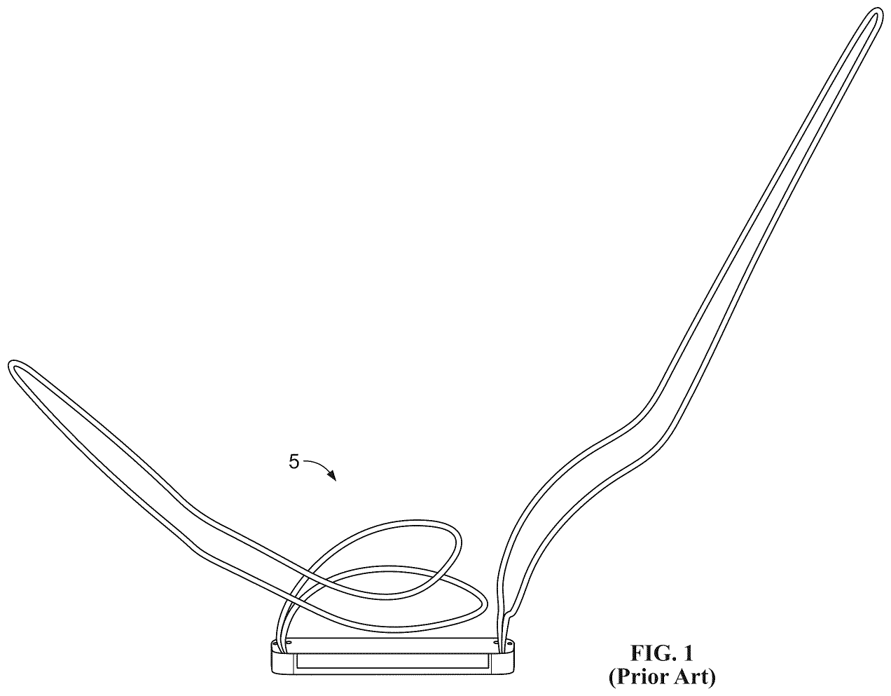

Turning to FIGS. 1-5, shown are embodiments of a wireless sensor for implant, a wireless reader, and systems thereof. For example, FIG. 1 shows an exemplary wireless sensor 5 known in the art for implant and positioning in a patient's pulmonary artery to measure pressure. The wireless sensor 5 may be permanently implanted, in an example.

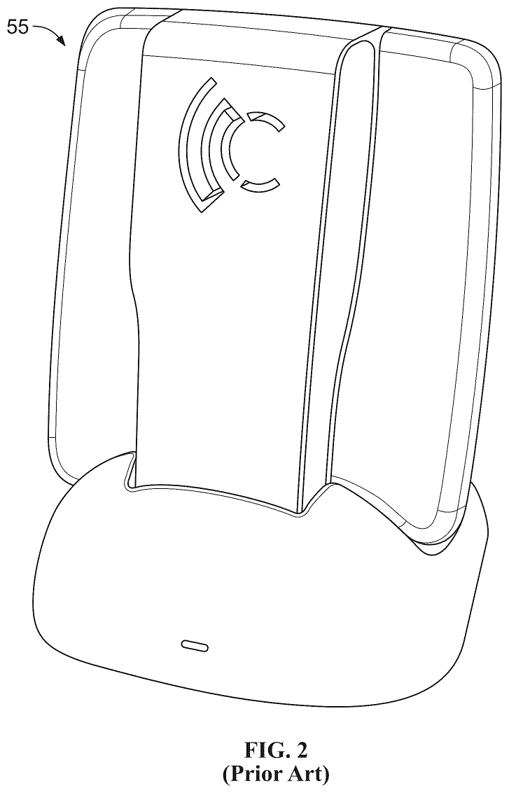

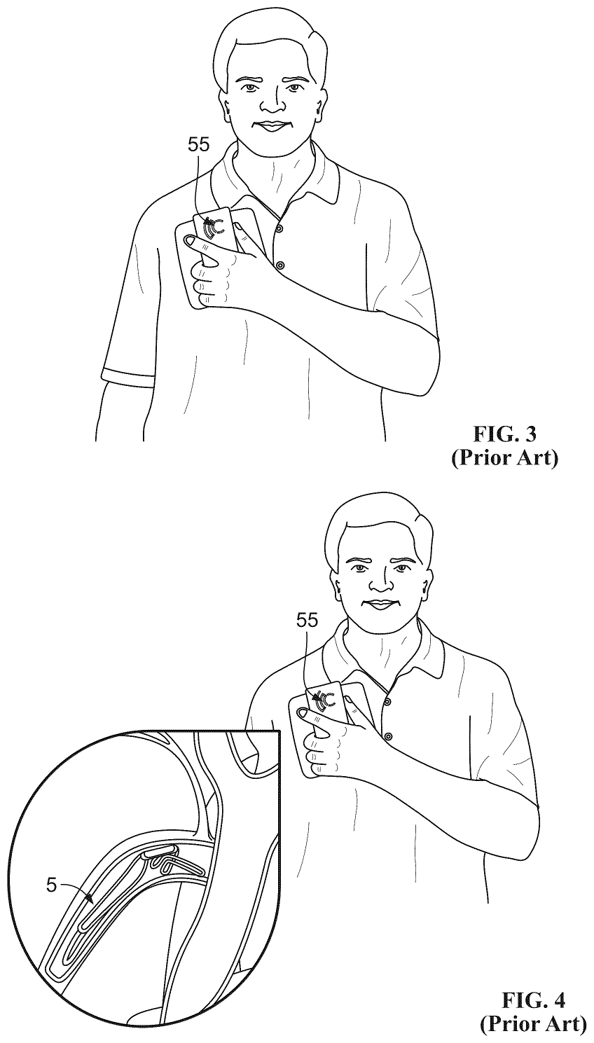

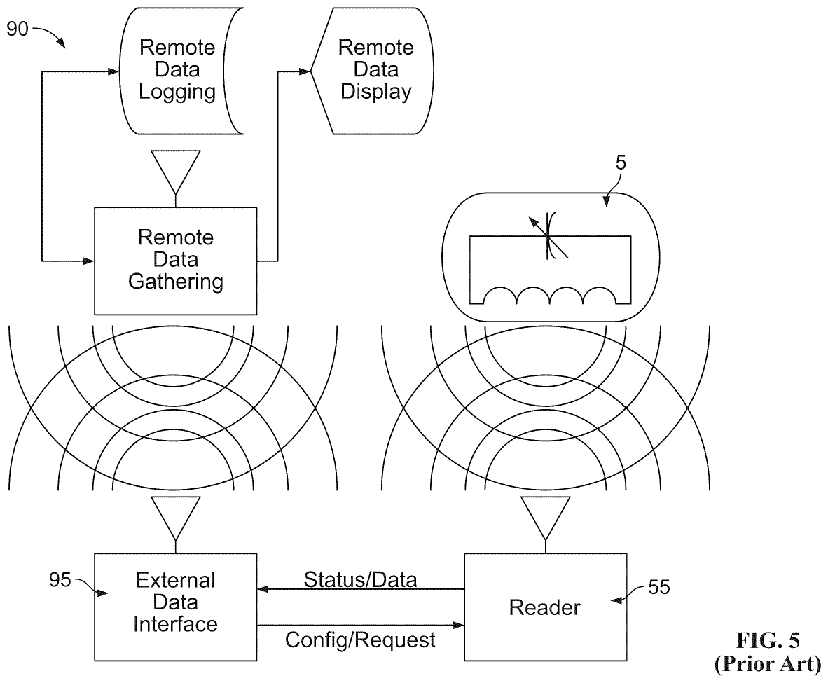

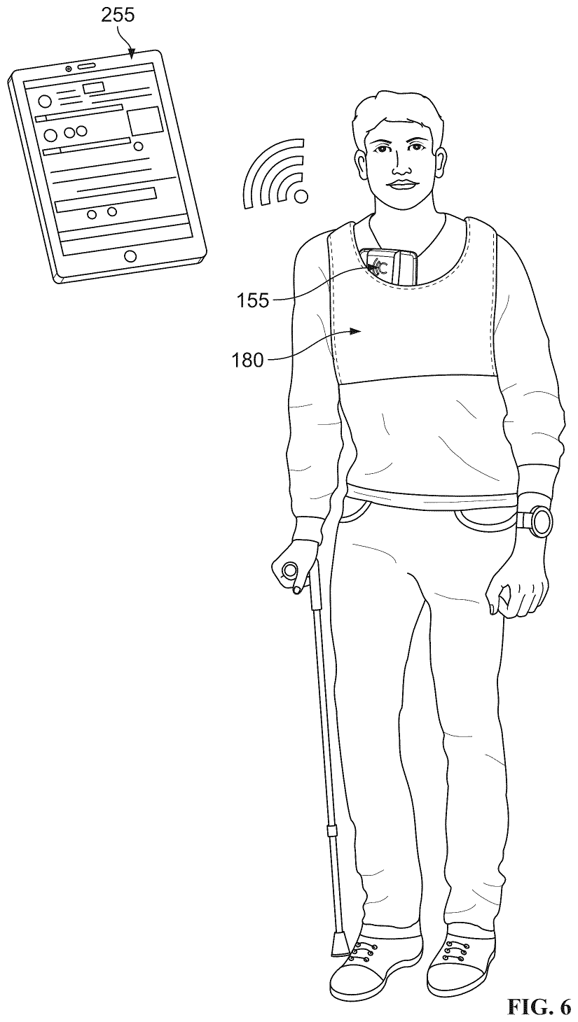

FIG. 2 shows an exemplary wireless reader 55 known in the art for coupled use with an implanted wireless sensor, such as wireless sensor 5 shown in FIG. 1 (see FIG. 4, for example). The wireless reader 55 may be used to receive and read pressure measurements from the implanted wireless sensor 5. The wireless reader 55 is a handheld device and, as shown in FIGS. 3-4, must be held in place on the right side of the patient's chest by the patient. wireless reader 55 must be placed in certain proximity to the implanted wireless sensor 5 and against the body of the patient. This position of the wireless reader 55 must be held and maintained by the patient in order for the wireless reader 55 to function and communicate with the implanted wireless sensor 5. Any movement of the wireless reader 55 may affect measurements and result in inaccurate or unreliable measurements. FIG. 5 shows an embodiment of an exemplary wireless sensor system 90 known in the art including an external data interface 95.

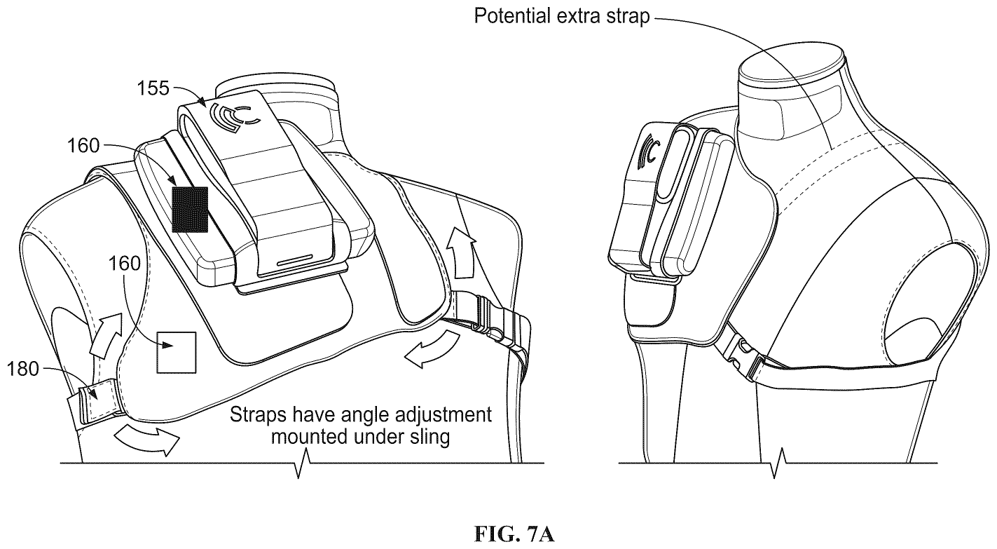

Turning to FIGS. 6-9, shown are embodiments of a reader module 155. The reader module 155 may be provided as part of a system 100 further including one or more (or all of) an implantable sensor 105, a data communication system 200, and a user device 255. The reader module 155 may be configured to communicate with and receive data from the implantable sensor 105 when in proximity of the implantable sensor 105. The reader module 155 may be a hands-free device as described in more detail below. The implantable sensor 105 may communicate with and transmit data to a user device 255, cloud network 205, or central hub for analysis by the data communication system 200 or a user, such as a doctor to determine the patient's heart health and associated conditions. The devices and systems may assist in managing patients with chronic heart failure or other health conditions. The devices and systems may be incorporated with other sensors and reading devices to generate more information regarding the health status of the patient. An example of such additional systems is found in U.S. Pat. No. 11,622,684, which is incorporated herein by reference. The systems disclosed in the '684 patent may be incorporated with the devices and systems of the present disclosure to provide this additional information on the patient.

The reader module 155 and system 100 may be able to record measurements and take a reading from the implantable sensor 105 requiring no other device aside from the reader module 155 and the implantable sensor 105. The reader module 155 may be hands free, portable, and may be able to be worn my a patient for extended periods of time (e.g., 30 minutes, 1 hour, more than 1 hour, several hours, 1 day, multiple days, and the like). The reader module 155 may be used with one hand or no hands and may take readings in a short period of time (e.g., less than 2 minutes, less than 1 minute, and the like.) The reader module 155 may be able to record measurements and take a reading from the implantable sensor 105 throughout various patient activities such as exercise, sleeping, moving (walking, jogging or running), and the like. The reader module 155 may be able to record measurements and take a reading from the implantable sensor 105 without requiring that the patient be in a certain position, e.g., only in an upright position or only in a supine position. The reader module 155 may be able to record measurements and take a reading from the implantable sensor 105 when the patient is in any position, in both upright and supine positions, in more than one position, when the patient is walking, standing, or laying down, when the patient is moving from upright to supine positions, or other positions, vice versa, and the like. The patient's position and movements need not be restricted in order for the reader module 155 to be able to record measurements and take a reading from the implantable sensor 105.

The reader module 155 and system 100 may be able to analyze the record measurements and readings from the implantable sensor 105 and adjust the data based on the activity or the patient, the position of the patient, and the like, to evaluate the data, provide trends, and assess patient health. The reader module 155 may be configured to selectively couple with the implantable sensor 105. For example, the reader module 155 may be able to recognize when the patient is in a supine position and that that the measurements are taken when the patient is in a supine position. For example, the reader module 155 may be able to recognize when the patient has moved from a supine position to an upright position and recognize that the measurements are now taken when the patient is in an upright position. Different patient positions and patient activity levels may lend to different measurement trends (for example higher heart rate when exercising). The reader module 155 and system 100 may be able to analyze the raw data from the implantable sensor 105 and may be able to implement a “correction factor” or adjustment based on patient positions and activity levels when the measurements were taken to evaluate underlying trends related to the patient's heart or health. The correction factor or adjustment may be based on collective trends applicable to most patients or the correction factor or adjustment may be patient specific. For example, the reader module 155 and system 100 may be calibrated to the patient. For example, the reader module 155 and system 100 may record baseline values of the patient when the patient is in different positions or during different activity levels so that patient-specific trends can be determined and applied to the raw data. It is noted that the described analyses may be carried out by the reader module 155 and system 100 or in a cloud network 205 or central hub where the raw data may be uploaded.

In an embodiment, the implantable sensor 105 may be implanted within a patient's body, e.g., within the patient's pulmonary artery, to measure pressures related to that location, e.g., pulmonary artery pressures or PAPs. Although reference is made to the implantable sensor 105 as implanted within the pulmonary artery, it is noted that any implanted wireless sensors may be used and the wireless sensors may be implanted in different locations as desired. The implantable sensor 105 may be implanted into a patient's circulatory system. Generally, the implantable sensor 105 may include a pressure sensor, electronics, a power source such as a battery, and an antenna. The implantable sensor 105 may measure pressure inside of the implanted location. In an embodiment, the pressure measurements may be taken continuously, at certain programmed intervals, manually through user input, or in response to predetermined conditions, such as recognition of the reader in a reading position against the patient's body. The electronics may include a processor, memory component, transmitter, and related circuitry in order to process and store pressure data obtained from the pressure sensor and to transmit the pressure data to the reader module 155. In an example, the implantable sensor 105 may also include an LC resonant tank, may be able to receive wireless power from the reader module 155, and the like. The antenna may facilitate the wireless communication and transmission of data between the implantable sensor 105 and the reader module 155. The implantable sensor 105 may comprise the sensor disclosed in U.S. Pat. No. 11,707,230, which is incorporated herein by reference in its entirety, the sensor disclosed in U.S. Pat. No. 11,547,320, which is incorporated herein by reference in its entirety, or the sensor disclosed in U.S. Pat. No. 10,638,955. Further, the implantable sensor 105 may be as described in U.S. Pat. Nos. 10,206,592, 10,993,669, 10,638,955, 10,226,218, 10,430,624, 10,003,862, 9,489,831, 9,721,463, 9,305,456 and 8,432,265 which are all incorporated herein by reference

In an embodiment, the reader module 155 may be configured to selectively interact or communicate with one or more devices, such as the implantable sensor 105. In an embodiment, the reader module 155 may be configured to selectively interact or communicate with two or more devices, such as the implantable sensor 105 and one or more user devices 255. The reader module 155 may be configured to record measurements from the implantable sensor 105 or other sensors. The user devices 255, for example, may be remote device and may include one or more (or all) of: a cellular phone, cellular or software application, a wearable device such as a fitness device, smart watch, or other activity tracker, computer, tablet, applications thereof, and the like. The reader module 155 may be configured to upload data to a cloud network 205 or central hub, or directly to an electronic health record or patient management data system. The reader module 155 may be configured to receive input parameters from the data communication system 200 to provide analyzed data and make a determination. The reader module 155 may be of any appropriate configuration. Some suitable configurations of the reader module 155 may comprise the reader assembly disclosed in U.S. Pat. No. 11,461,568, the sensor reader in U.S. Pat. No. 9,894,425, the sensor reader in U.S. Pat. No. 10,003,862 or the sensor reader in U.S. Pat. No. 8,493,187 all of which are incorporated herein by reference in their entirety.

Generally, the reader module 155 may include electronics, a power source such as a battery, rechargeable battery, and/or power adapter, and an antenna. The electronics may include a processor, memory component, receiver and transmitter (or transceiver), and related circuitry in order to process and store pressure data obtained from the implantable sensor 105 and to transmit the pressure data to the user device 255, cloud network 205, or central hub. In an embodiment, the reader module 155 may include an integrated display screen. In an embodiment, the reader module 155 may selectively connect to an external display screen, user device 255 having a display screen, or to a cellular or software application having display capabilities. The display screens may display live or historical recorded data, program settings, alerts, and the like. In an embodiment, the reader module 155 may include a user interface that can be used to control the reader module 155, access data, change device settings, and the like. In an example, the reader module 155 may be manually activated (e.g., by a button, gesture, voice, fingerprint, etc.) for impromptu readings and to start and/or stop reading timeframes. It is noted that timed readings may also be taken where the reader module 155 is manually activated to start a reading and the reading stops automatically after a set amount of time. Reading timeframes may also be started or stopped automatically based on a variety of factors, including but not limited to, time, patient position, activity, one or more predetermined parameters, and the like. For example, the reader module 155 may be left in an “on” or “wake” state and may be programmed to automatically take periodic short readings. The reader module 155 may be programmed to take 20 second readings every couple hours, for example, may be programmed to take readings at certain times of day, may be programmed to take readings when it detects exercise, sleep, or a change in patient position, and the like.

Communication between the reader module 155 and the implantable sensor 105 may be facilitated by a short-range communication system, such as near field or mid-field communication systems. For example, the antenna of the reader module 155 may facilitate the wireless communication and transmission of data between the reader module 155 and the implantable sensor 105, and may recognize and interact with the antenna of the implantable sensor 105. In an embodiment, the implantable sensor 105 may transmit raw data to the reader module 155.

Communication between the reader module 155 and user device 255 may be facilitated by a wireless communication framework that allows for all-range communications. In an embodiment, the reader module 155 may communicate with the user device 255 through a communication framework, such as Wi-Fi, Bluetooth, Zigbee, cellular networks, medical implant communication systems (MICS), wired connection, and the like. For example, the reader module 155 may include connectivity options to transfer patient data to a computer system, electronic health record, or patient management data system, which can allow direct and remote access to the data by a patient's health care team. In an embodiment, the reader module 155 may interact with a cloud network 205 or central hub to upload data and information which can later be downloaded by one or more approved devices, such as user devices 255. In an embodiment, the reader module 155 may transmit raw data to the user device 255, cloud network 205, or central hub. In an embodiment, the reader module 155 may analyze the raw data and transmit analyzed data to the user device 25, cloud network 205, or central hub.

In an embodiment, the reader module 155 may be configured to communicate with another medical device. For example, the reader module 155 may be configured to communicate with a pulse oximeter, implanted pacemaker, electrocardiogram, continuous positive airway pressure (CPAP) machines, etc. In an embodiment, the reader module 155 may be able to directly communicate with the medical device, for example, by Wi-Fi, Bluetooth, Zigbee, cellular networks, medical implant communication systems (MICS), wired connection, and the like. In an embodiment, the reader module 155 may be able to indirectly communicate with the medical device through the cloud network 205 or central hub. For example, both the reader module 155 and the medical device may be able to receive and transmit information such as instructions or reading data to the cloud network 205 or central hub, which can then be accessed by the other device.

In an embodiment, the reader module 155 may be triggered to take a reading on command from the medical device based on the medical device's recognition or detection of a certain event or parameters. For example, a continuous positive airway pressure (CPAP) machines may instruct the reader module 155 to take a reading and record pulmonary artery pressures when an apnea event is recognized or detected (by the CPAP machine). In an embodiment, the reader module 155 may be triggered to take a reading based on its own (or the system's 100) analysis of the data to recognize or detect a certain event or parameters and/or may instruct a medical device to take a reading when certain criteria are met. For example, the reader module may recognize or detect a change in the patient's position or activity (e.g., exercising or sleeping) and may initiate readings from the implantable sensor 105 related to pulmonary artery pressure, in an example, and/or may instruct a pulse oximeter to take a reading and record blood oxidation levels.

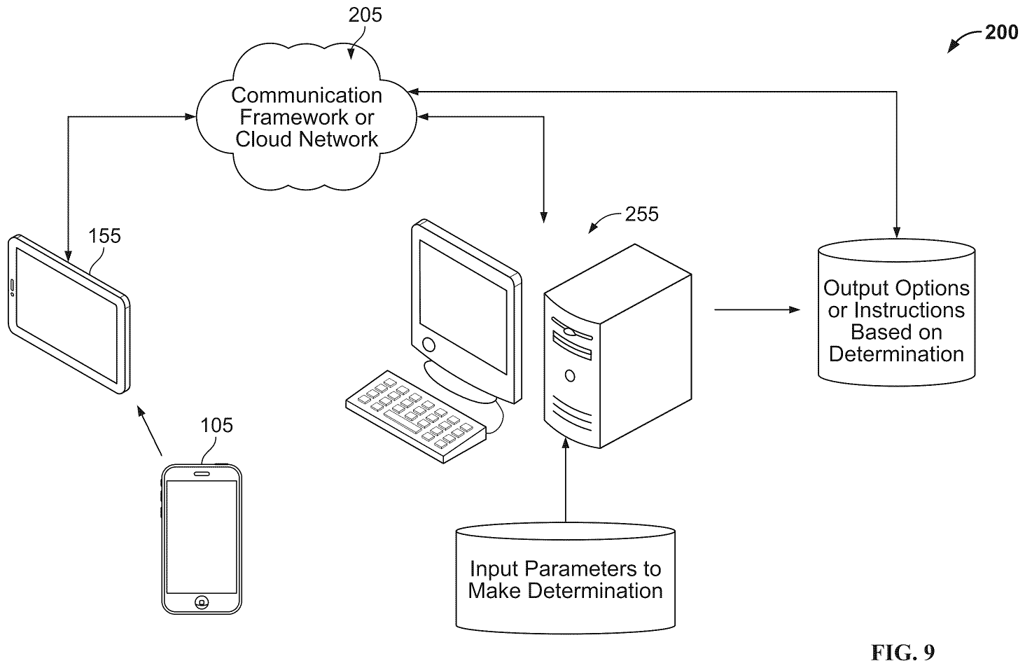

As shown in FIG. 9, the data communication system 200 may relay one or more input parameters to the data communication system 200, which may analyze the raw data to make a determination, and which may output options or instructions based on the determination. It is noted that the input and output may be facilitated through the reader module 155, through the user device 255, or through a combination thereof. In an embodiment, the implantable sensor 105, reader module 155 and system 100 data may be coupled with treatment algorithms that can analyze the raw or analyzed data and diagnose the mechanisms, stages, and characteristics of congestion in the patient and guide appropriate treatments and interventions.

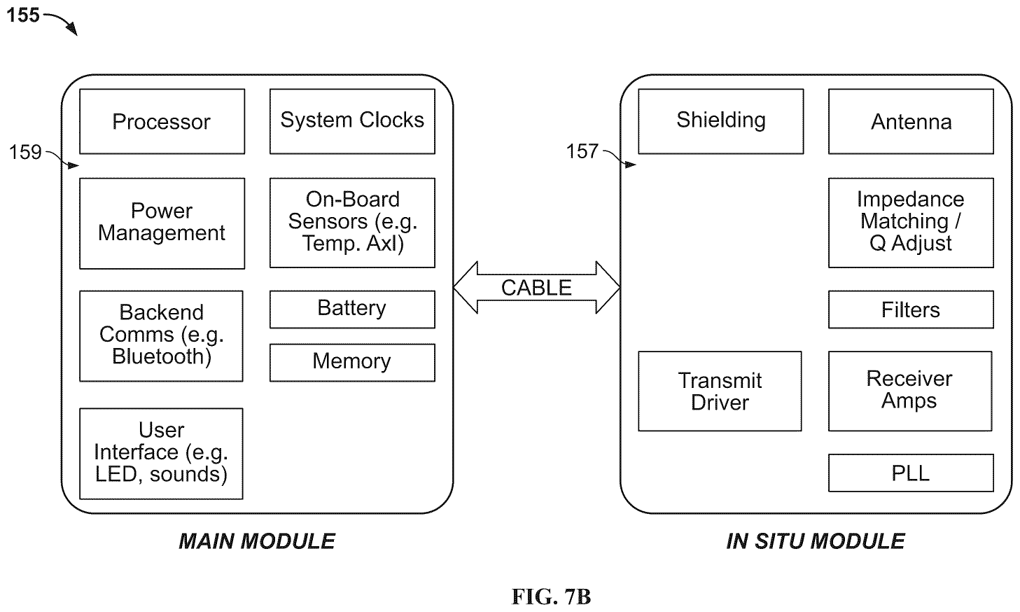

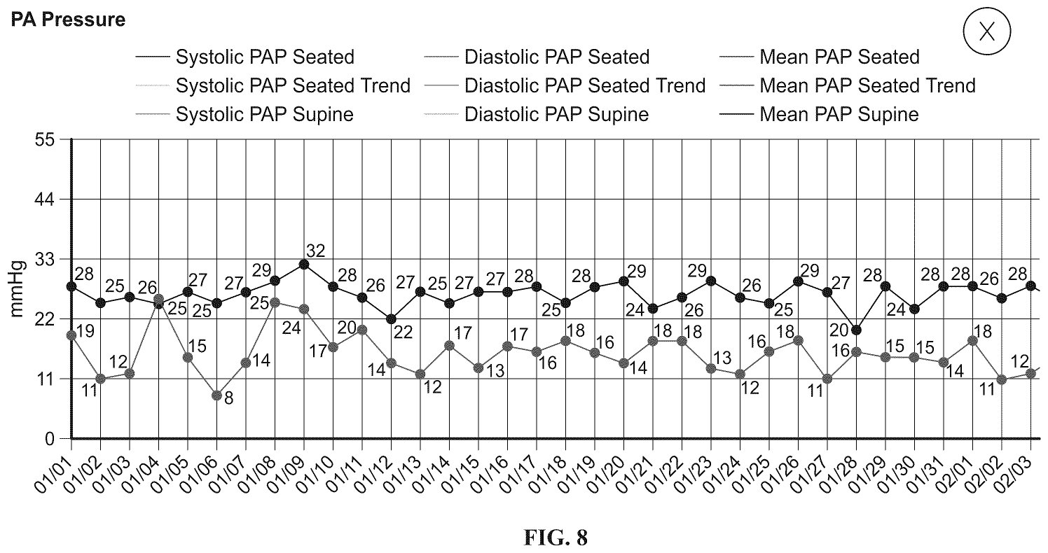

In an embodiment, the reader module 155 may include one or more sensors 160. In an embodiment, the reader module 155 may include a sensor 160 to determine patient position. It is noted that the sensor 160 may alternatively or additionally be positioned on the garment 180, see FIG. 7A showing sensors on the reader module 155 and garment 180. In an embodiment, the reader module 155 may include a motion sensor or a plurality of motion sensors of any appropriate configuration. In an embodiment, the reader module 155 may include an accelerometer, velocity sensor, gyroscope, pressure sensor, or tilt sensors for detecting orientation of the reader module 155 with respect to gravity. The accelerometer or tilt sensor may be able to detect the posture and position of a patient and changes thereof. The accelerometer or tilt sensor may sense the reader modules 155 orientation with respect to gravity, and may infer or determine the orientation of the patient's torso (upright or decubitus, i.e. seated or supine). FIG. 8, for example, shows a graphical representation of longitudinal data display of parameters obtained in seated and supine positions by the reader module 155.

The accelerometer may be able to measure other data related to the patient, such as steps take, distance walked, speed of the walk, and the like. In other embodiments the motion sensor may be a separate device from the reader module 155. In such embodiments, the motion sensor may be wirelessly or directly wired to the reader module 155 and provide data on motion of the patient. Such motion sensors may comprise passive infrared sensors, ultrasonic sensors, microwave sensor, tomographic sensor or the like. Alternatively or in addition, a motion sensor or a plurality of motion sensors (such as an accelerometer or tilt sensor) may be incorporated into the garment 180 and may be used to detect the posture, position and changes thereto of the patient.

Generally, the reader module 155 (and/or garment 180) may be able to automatically detect and classify the posture and position of a patient to correlate the posture and position of the patient with the measured pressures by the implantable sensor 105 to provide correlated data. In an embodiment, the reader module 155 may provide a visual, audio, or haptic cue to the patient and require a patient response (e.g. button push or spoken acknowledgement) to manually select the patient's position, activity level, etc. The reader module 155 may also include a camera, internal magnetometer or gyroscope, a tilt sensor in the form of a ball switch or spring/mass, etc., to detect the posture and position of a patient and changes thereof. In an embodiment, the reader module 155 may be configured to not start the reading until one of these sensors confirms the patient is a specific position and/or the patient manually confirms the patient's position.

The reader module 155 may include other sensing, communication, connectivity, and/or data components, including, for example, one or more (or all) of the following: a GPS or other capability to record location; communication capability to other wearable devices such as fitness devices, smart watches, or activity trackers; communication capability to other smart devices such as a cellular phone, computer, tablet, or the like; medical device components such as an electrocardiogram, pulse oximeter, optical heart sensor, stethoscope for heart or lung sounds, body temperature thermometer, or the like; microphone or recorder to recognize and provide response to voice comments and to record voice notes related to appointments, healthcare instructions, or monitoring of symptoms such as symptomatic arrhythmia or when chest pain occurs, for example; capability to record dates and time; capability to determine and record ambient pressure and/or temperature; capability to collect catheter pressure measurements e.g., through an integrated pressure sensor that is attachable to a catheter or through an analog or digital channel; capability to serve as a hub for other devices the patient may have such as an electrocardiogram, pulse oximeter, optical heart sensor, stethoscope for heart or lung sounds, body temperature thermometer, blood pressure cuff, scale, or the like; communication capability to connect to another device or other related network to alert clinician, family, or emergency services when certain parameters are met, e.g., to indicate a fall, heart attack, severe heart failure symptoms, stroke, or the like; alert capability to alert the patient when certain parameters are met, e.g., to indicate a fall, heart attack, severe heart failure symptoms, stroke, or the like; integrated application specific integrated circuits (ASIC) implementation for portability during exercise; communication capability or integrated an Augmented Reality (AR) system to provide instructions or information to the user; etc.

It is noted that any of the foregoing described communication or connectivity capabilities may be facilitated by one or more (or all) of: Wi-Fi, Bluetooth, Zigbee, cellular networks, medical implant communication systems (MICS), wired connection, etc. It is noted that the reader module 155 or other component of system 100 may have communication capability with a cellular or software application, cloud network 205, central hub, or directly to an electronic health record or patient management data system. It is noted that the reader module 155 may be incorporated, integrated with, or provided as a part of another smart or electrical device, such as a cellular phone, computer, tablet, other wearable devices such as fitness devices, smart watches, or activity trackers, or the like.

The correlated data may similarly be coupled with treatment algorithms that can diagnose the mechanisms, stages, and characteristics of congestion in the patient and guide appropriate treatments and interventions. The reader module 155 and system 100, being able to distinguish the patient's position and provide correlated data based on the patient's position, allows the reader module 155 and system 100 to operate and provide reliable and accurate data regardless of the patient's position. For example, pressure measurements and reader module operation are not limited to certain positions of the patient, but rather the reader module 155 and system 100 can operate and provide analyzed pressure measurements of the patient in any position, including supine or decubitus positions, prone positions, and upright positions such as being seated, standing, walking, etc., and any other positions such as recumbent positions, and the like. In an embodiment, the patient may be able to move between various positions while the reader module 155 is attached to the patient and receiving data from the implantable sensor 105.

The reader module 155, or component module thereof, may be secured to the patient by a garment 180, such as a strap, holster, girdle, vest, shirt, or the like. The garment 180 may be generally tight-fitting around the patient's chest. In an embodiment, the garment 180 may include adhesives, pockets, hook and loop fasteners, buttons, magnets, or the like, that correspond and attach to mating mechanisms on the reader module 155 to direct the reader module 155 into a desired position against the patient related to the implantable sensor 105 implanted within the patient. In an embodiment, the reader module 155 may give visual, audio, or haptic feedback to help patient locate the implantable sensor 105 and/or confirm correct placement of the reader module 155 relative the implantable sensor 105.

In an embodiment, an adhesive may be used alternatively or in addition to garment 180 and may selectively attach the reader module 155 to the body (such as the skin) of the patient to hold the reader module 155 in place. The garment 180 may include an elastic band and include generally stretchable or other accommodating material. In an embodiment, the garment 180 may wrap around a patient's upper torso or ribcage. In an embodiment, the garment 180 may wrap around one or both of the patient's shoulders, e.g., at least the patient's right shoulder in pulmonary artery pressure applications. The reader module 155 may be placed between the garment 180 and the patient's body and may be held in place by the garment 180. The garment 180 may prevent the reader module 155 from unintentionally moving even as the patient moves positions and may allow for hands free operation of the reader module 155. This may even allow the patient to exercise while wearing the garment 180.

The reader module 155 and/or garment 180 may be suitable for a variety of patient states and positions, including exercise, sleeping, and the like, and can generally move with the patient's body so that accurate measurements may be taken and received throughout any movement of the patient or changes of the patient states and positions. The reader module 155 and/or garment 180 may be suitable for long term wear and monitoring. Another embodiment of such reader module 155 and garment 180 is disclosed in US Patent Pub. No 20220211282, which is incorporated herein by reference in its entirety.

In an embodiment, the reader module 155 may be provided as a single device that is selectively configured to be placed in proximity to the implantable sensor 105, e.g., on the patient's body, see FIGS. 6-7A, for example. As shown in FIG. 7B, the reader module 155 may be provided in two (or more) modules. For example, the reader module 155 may be provided in two modules, such as an in-situ module 157 and a main module 159. In an embodiment, the two (or more) modules may separate components of the reader module 155 so that bulkier components may be located in a module not bound to the patient's skin or chest and the component that is positioned on the patient's skin or chest to interact with the implantable sensor 105 can be slimmed down and more easily wearable. In an embodiment, the reader module 155 or component thereof (e.g., in-situ module 157 or main module 159) may be a flexible pad.

In an embodiment, the in-situ module 157 may be worn near or in proximity to the implantable sensor 105 (like the singular reader module 155 device). In an embodiment, the main module 159 may be worn or attached to the patient in a position on the patient that is indifferent to the location of the implantable sensor 105. For example, the main module 159 may be placed on a belt or in a pocket of the patient, may be attached to an arm strap, or may be positioned anywhere else on the patient as may generally be comfortable or desired by the patient. The in-situ module 157 and the main module 159 may connect or communicate through a wired cable connection. It is noted that the in-situ module 157 and the main module 159 may connect or communicate through a wireless connection.

The in-situ module 157 may include components of the reader module 155 such as an antenna, shielding, filters, transmit driver, transceiver circuit, receiver amps, impedance matching and quality adjustment components, phase locked loop circuits, on-board sensors such as temperature sensors and accelerometers, and the like. In an embodiment, the in-situ module 157 may include only the antenna. In an embodiment, the in-situ module 157 may include only the antenna and transceiver. In an embodiment, the in-situ module 157 may include only the antenna, transceiver, and a battery. The main module 159 may include components of the reader module 155 such as a processor, system clocks, power management, on-board sensors such as temperature sensors (e.g., to determine ambient and/or body temperature) and accelerometers, backend communications such as Wi-Fi, Bluetooth, Zigbee, cellular networks, medical implant communication systems (MICS), wired connection, etc., battery memory, and user interface including, in an example, LED or sounds, and the like. It is noted that these described components of the in-situ module 157 and the main module 159 may also be provided in a single device reader module 155.

Generally, the in-situ module 157 may include components necessary to communicate with or interact with the implantable sensor 105 (e.g., that require a “fixed” position within the system 100 and precise attachment location on the body of the patient near the implantable sensor 105) and the main module 159 can include any bulkier or other components that do not need to be in direct proximity to the implantable sensor 105 (e.g., that do not require a “fixed” position within the system 100 and that only require being moveable with the patient's movement, e.g., being attached to the patient body, but that do not require precise attachment location on the body of the patient). In an embodiment, a portable module may include components that have front end uses (e.g., circuits needed to do the portable measurements and a battery) and a stationary dock may be provided to include components that have back end functions (e.g., data upload, data storage, processing, and the like).

In an embodiment, the in-situ module 157 may be provided as an adhesive sticker or pad. The adhesive sticker or pad may be single use or disposable. The adhesive sticker or pad may comprise the in-situ module 157 or may attach the in-situ module 157 to the patient directly (e.g., at the patient's chest corresponding to the location of the implantable sensor 105). The in-situ module 157 having an adhesive sticker or pad may not require a garment 180 for securement of the in-situ module 157 to the patient. In an embodiment, the adhesive sticker or pad may include a thin and/or flexible battery. In an embodiment, the in-situ module 157 may be provided as an integrated component within a tight-fitting shirt, such as an exercise top. In an embodiment, in-situ module 157 as a shirt may include the antenna integrated in the shirt and the shirt may include a connector for the main module 159.

Unless this disclosure or context suggests otherwise, the “supine” position may be generally understood as any recumbent or decubitus position, including supine, lateral decubitus, or prone. Unless this disclosure or context suggests otherwise, the “seated” position may be generally understood as any upright position, including standing, sitting, walking, or kneeling. All aspects of this disclosure may be applied to postural positions between upright and recumbent as well, for example a full recumbent posture measurement could be compared to a 45 degree reclining posture measurement instead of a full upright posture.

In an embodiment, a method for assessment and management of congestion in heart failure may include one or more (or all) of: implanting within a patient with an implantable sensor, such as implantable sensor 105, that measures at least one hemodynamic parameter, such as pressure and pulmonary artery pressure; providing a portable or wearable external reader module, such as reader module 155, configured to communicate with the implantable sensor when the patient is in a specific state or position or when the reader module is placed in a specific state or position, e.g., near the implantable sensor and/or within the corresponding garment, such as garment 180. The method may include operating the reader module when the patient is in a first state or position, in order to acquire data from the implantable sensor related to the least one hemodynamic parameter. The method may include operating the reader module when the patient is in a second state or position, in order to acquire data from the implantable sensor related to the at least one hemodynamic parameter. The methods may estimate a postural response based on the hemodynamic parameter(s) measured in the first and second state or position. In an example, the first state or position is the patient in a supine position and the second state or position is the patient in a seated position, or vice versa. In an embodiment, the methods may include acquiring posture information from motion monitoring sensors and associated algorithms.

In an example, the estimated postural response may be determined as the difference in pulmonary artery pressures in the supine and seated positions. The reader module 155 may also be able to evaluate other parameters of interest between seated and supine positions, including one or more (or all) of: heart rate; pulmonary artery pulse pressure; area under the pressure curve; pressure rise and fall times; arrhythmias; respiratory rate; respiratory amplitude; dicrotic notch location on waveform, and the like. In an example, the estimated postural response may include comparing the postural response with a reference value. In an embodiment, the reference value may be a baseline value measured at a given time. In an embodiment, the reference value may be measured in a clinical setting in combination with other parameters. Such other parameters may include, in an example, stroke volume, ejection fraction, and other things that one can measure accurately in a clinical setting. In an example, the postural response may be indicative of one or more (or all) of: a change in patient's body blood volume distribution; a change in the patient's venous capacity; a change in the patient's venous compliance and/or resistance; a change in the patient's volume status; a change in the patient's ejection fraction status; a change in the patient's disease severity; a patient's status and type of pulmonary hypertension, and the like. In an embodiment, the postural response may be measured in combination with one or more (or all) of the patient's heart rate, pulse oxidation, or pulse pressure. In an embodiment, the postural response may be measured in combination with one or more (or all) of spirometric parameters such as vital capacity (VC), forced vital capacity (FVC), forced expiratory volume (FEV) at timed intervals of 0.5, 1.0 (FEV1), 2.0, and 3.0 seconds, forced expiratory flow 25-75% (FEF 25-75) and maximal voluntary ventilation (MVV), also known as maximum breathing capacity.

The method may include uploading the data to an external device, such as a user device 255, cloud network 205, or central hub. The method may include assessing and treating the patient based on the measured estimated postural response and other hemodynamic parameter(s) and and/or any trends thereof.

In an embodiment, a system 100 for assessment and management of congestion in heart failure may include one or more (or all) of: a wireless implantable sensor that measures a hemodynamic parameter, such as implantable sensor 105; a wireless external reader that is configured to communicate with the implantable sensor, such as reader module 144. In an embodiment, the reader may have a small, portable form. In an embodiment, the reader may be battery powered. In an embodiment, the reader may be provided in a wearable configuration.

In an embodiment, the hemodynamic parameter may be measured in at least two patient states and/or positions. In an embodiment, the system may estimate a postural response by comparing measured hemodynamic parameters in two or more patient states and/or positions. In an embodiment, the first state or position may be the patient in a supine position and the second state or position may be the patient in a seated position (or vice versa). In an embodiment, the system may acquire posture information from motion monitoring sensors and associated algorithms.

In an embodiment, the estimated postural response may be understood as the difference in pulmonary artery pressure in the supine and seated positions. In an example, the estimated postural response may include comparing the postural response with a reference value. In an embodiment, the reference value may be a baseline value measured at a given time. In an embodiment, the reference value may be measured in a clinical setting in combination with other parameters. Such other parameters may include, in an example, stroke volume, ejection fraction, and other things that one can measure accurately in a clinical setting. In an example, the postural response may be indicative of one or more (or all) of: a change in patient's body blood volume distribution; a change in the patient's venous capacity; a change in the patient's venous compliance and/or resistance; a change in the patient's volume status; a change in the patient's ejection fraction status; a change in the patient's disease severity; a patient's status and type of pulmonary hypertension, and the like. In an embodiment, the postural response may be measured in combination with one or more (or all) of the patient's heart rate, pulse oxidation, pulse pressure. In an embodiment, the postural response may be measured in combination with one or more (or all) of spirometric parameters such as vital capacity (VC), forced vital capacity (FVC), forced expiratory volume (FEV) at timed intervals of 0.5, 1.0 (FEV1), 2.0, and 3.0 seconds, forced expiratory flow 25-75% (FEF 25-75) and maximal voluntary ventilation (MVV), also known as maximum breathing capacity.

As described herein, the implantable sensor may be implanted into the cardiovascular system to measure hemodynamic parameters. It is noted that the implantable sensor or other sensors may be implanted into the central venous system or the splanchnic veins, either in combination with the pulmonary artery sensor or alone. For example, a sensor may be implanted in at least one of: the pulmonary artery, right atrium, hepatic vein, portal vein, splanchnic veins, inferior vena cava, superior vena cava, brachial vein, and the like. Additionally, the methods and systems may be combined with at least one other diagnostic selected from: ultrasound, CT scan, fluoroscopy, catheter-based sensor, heart sounds, lung sounds, electrocardiograms, arterial blood pressures, pulse oximetry, blood samples, glomerular filtration rate measurement, B-type natriuretic peptide levels, thoracic impedance, and the like.

The patient may be in any state and position during measurements taken by the implantable sensor 105. For example, patient states may be before, during, or after one or more exercises and/or activities, sleep, feeling symptomatic (e.g. arrhythmia, dyspnea, chest pain, numbness, palpitations, etc.) The patient stat can also include being attached to a life-supporting or other medical machine, such as dialysis, extracorporeal membrane oxygenation (ECMO) machine, blood transfusion, ventilator, oxygen cannula, chemotherapy, external pacemaker, nasogastric or orogastric tube, neural electrodes, epidural or other drip, peripherally inserted central catheters (PICC) lines, arterial lines, chest tubes, electrocardiograms, etc.

The patient state can also include any situation where it may be advantageous to have a wearable reader such as situations that could otherwise be difficult to bring the patient into contact with a larger or more cumbersome reader device. For example, patient states can include when the patient is performing activities, exercising, unconscious, comatose, obese, frail, immobile, mentally impaired, paralytic, palsied, restrained, sedated, undergoing surgery, or in cases where movement causes pain or difficulty such as muscular diseases, arthritis, atrophy, burns or skin irritation, etc., and the like.

The system 100, implantable sensor 105, and/or reader module 155 may track and output one or more (or all) of the following measurement quantities: systolic, diastolic, mean pulmonary artery pressure, and pulmonary artery pulse pressure; pulmonary artery waveforms including transit time, systolic time, pulmonary pressure at the inflexion point, augmentation index, pulse wave velocity, rise and fall time, dicrotic and anacrotic notch locations (valve closure), area under curve, cardiac output estimation; heart rate and breathing rate (e.g., from the pulmonary artery waveform); pulmonary artery changes due to breathing (surrogate spirometry), including minimums, maximums, rates, and derivative of pressure over time (dP/dt) during inhale and exhale; sPAP during exercise (highest); dPAP during sleep (lowest of the day), and the like.

The system 100, implantable sensor 105, and/or reader module 155 may track and output one or more (or all) of the following derived parameters: cardiac output, stroke volume, cardiac index, stroke volume index, total pulmonary resistance, systemic and vascular pulmonary resistance, arterial and vessel compliance, resistance, and stiffness, full right heart functionality, including heart failure with reduced ejection fraction (HFrEF) and preserved ejection fraction (HFpEF) of the right heart, and the like.

The system 100, implantable sensor 105, and/or reader module 155 may calculate and output one or more (or all) of the following: ratio of pulmonary artery pressures over cardiac output, slope of the change between different postures and states; diastolic pulmonary artery pressures during sleep (instead of or in addition to left ventricle filling pressure and/or central venous pressure); comparison of two or more pressure sensors in different locations such as one in the central venous system and one in the pulmonary artery; right ventricle delta-pressure and estimate right heart stroke volume, tricuspid valve function, regurgitation and right ventricle contractility; arterial pressure combination to evaluate left heart function (e.g., using pulmonary artery pressures plus a second implanted arterial side sensor or external device); comparison of systolic pulmonary artery pressures during exercise and rest or in upright and recumbent positions; pulmonary artery compliance or pulmonary vascular resistance (PVR) by pressure change during rest compared to exercise, or by comparing values at seated, supine, and other positions; comparison of pulmonary artery pressure or central venous pressure variations between different body positions (e.g., seated, supine, prone, left/right side, legs raised, head lower than torso, Valsalva maneuver, etc.); parameters (such as pressure) when exercising; comparison of measurements before, during, and after activity or stress tests; measurement of symptomatic arrhythmia on-demand; detect and record cough events (e.g., by a short, large pressure spike); detect and classify activities based on sensors and recorded data and correlate activities with hemodynamic parameters; changes in hemodynamic parameters between different activities and/or between rest and activities; dynamic and static arterial compliance; etc.

In an embodiment, system 100 and methods thereof may assess heart factors of a patient including volume level and compliance and/or resistance level by the following steps or algorithm: measure recumbent pressure reading at rest, measure upright pressure reading at rest, measure upright pressure reading after exercise. If the delta pressure is large between upright rest compared to upright exercise, the vessels of the patient may be considered to have good compliance. If delta pressure is small, the vessels of the patient may be considered to have poor compliance and/or high resistance (stiff or stenotic). If the vessels have poor compliance and/or high resistance and delta pressure is large between seated and supine positions, the patient may be considered as having fluid overload. Such pressure readings can be taken in the pulmonary artery, central venous system, or other vessels.

Other pressure values (including, e.g., waveforms, peaks and valleys, rates of change on fast and slow scales) as well as other measurable values may be tracked with implantable pressure sensors or other sensors implanted in anatomical zones in addition to or alternatively to the pulmonary artery. For example, other anatomical zones that may be utilized include, but are not limited to, central venous pressure, right atrial pressure, any locations within the central venous system; hepatic-related pressures in portal shunts or in the portal vein; throughout the gastro-intestinal tract, the kidneys, ureters, and bladder; the lungs (pre- and post capillary); the cranium (e.g., hydrocephalus, aneurysm, head trauma, etc.); the eyes (for example, to monitor or detect glaucoma); orthopedics, artificial knees & hips, prosthetics, etc. (for example, to monitor or detect strain sensing); related to transplanted organs (e.g., liver, heart, lungs, etc.) with implantation of a desired sensor pre-operatively, during transplant, or post-operatively; built into other implants (shunts, IVC filters, stents, artificial valves, etc.); in the splanchnic veins; and the like. Moreover, the system 100, implantable sensor 105, and/or reader module 155 may track and output longitudinal change over time of any of the foregoing.

The system 100, implantable sensor 105, and/or reader module 155 may provide a minimally invasive, hands free, at-home optional, small and non-cumbersome monitoring system that is useful for monitoring a patient during a variety of patient states including in one or more patient states (e.g., upright, supine, prone, during exercise, and the like).

The system 100, implantable sensor 105, and/or reader module 155 may be used or have application to one or more (or all) of the following: at a cardiology clinic, non-cardiology clinic (e.g., dialysis, chemotherapy or infusion, nursing home, etc.) or other hospital clinics or care settings (e.g., nursing open, physical therapy facilities); during surgical procedures to provide live and/or recorded pressures throughout the procedure; at home; during exercise or activity; during sleep; while at work, on travel, or doing errands; right before or right after taking certain medications; etc. In an embodiment, the reader module 155 may be portable so that the patient can take the reader module 155 with them throughout their daily activities to take manual measurements and use the reader module 155 on an as needed basis (e.g., kept in a purse car, locker, or the like). It is noted that the reader module 155 may be slim, comfortable, and hands free so that the patient can wear the reader module 155 throughout their daily activities to take programmed, scheduled or automatic measurements.

Although the embodiments of the present teachings have been illustrated in the accompanying drawings and described in the foregoing detailed description, it is to be understood that the present teachings are not to be limited to just the embodiments disclosed, but that the present teachings described herein are capable of numerous rearrangements, modifications and substitutions without departing from the scope of the claims hereafter. The claims as follows are intended to include all modifications and alterations insofar as they come within the scope of the claims or the equivalent thereof.

What is claimed is:

1. A system for assessment and management of congestion in heart failure, comprising:
an implantable sensor configured to insert into a circulatory system of a patient;
an external reader module configured to selectively couple with the implantable sensor and record measurements taken from the implantable sensor; and
an accelerometer within the external reader module, wherein the accelerometer is configured to detect a position of the patient and wherein the external reader module is configured to record at least one hemodynamic parameter when the patient is in a first position and in a second position and wherein the at least one hemodynamic parameter is compared between the patient being in the first position with a reference value and the patient being in the second position with the reference value.

2. The system of claim 1, wherein the first position comprises a supine position.

3. The system of claim 2, wherein the second position is an upright position.

4. The system of claim 1, wherein the external reader module is configured to record measurements when the patient is walking, exercising, standing, and sleeping.

5. The system of claim 1, wherein the external reader module is hands-free.

6. The system of claim 5 further including an adhesive configured to attach the external reader module to the patient.

7. The system of claim 5 further including a garment configured to attach the external reader module to the patient.

8. The system of claim 1, wherein the external reader module comprises a first module and a second module, wherein the first module is configured to attach to the patient near the implantable sensor and the second module is configured to attach to the patient anywhere on a patient's body.

9. The system of claim 8, wherein the first module includes only an antenna for near field or mid-field communication with the implantable sensor and an adhesive configured to attach first module to the patient.

10. The system of claim 1, wherein the external reader module is configured to compare the at least one hemodynamic parameter when the patient is in the first position against the at least one hemodynamic parameter when the patient is in the second position.

11. The system of claim 1, wherein the hemodynamic parameter comprises volume level of a vessel of the patient.

12. The system of claim 1, wherein the at least one hemodynamic parameter comprises a resistance level of a vessel of the patient.

13. The system of claim 1, wherein the first position is a supine position with the patient at rest and the second position is a seated position with the patient at rest, and wherein the at least one hemodynamic parameter comprises pressure.

14. The system of claim 13, wherein the external reader module is configured to record the pressure when the patient is in a third position, wherein the third position comprises a supine position with the patient exercising.

15. A system for assessment and management of congestion in heart failure, comprising:
an implantable sensor configured to insert into a circulatory system of a patient;
an external reader configured to selectively record measurements taken from the implantable sensor; and
an accelerometer within the external reader, wherein the accelerometer is configured to detect a position of the patient, wherein the external reader is configured to record a hemodynamic parameter when the patient is in a first position at rest, when the patient is in the first position at exercise and when the patient is in a second position at rest, and wherein the hemodynamic parameter of the patient being in the first position at rest and the hemodynamic parameter of the patient being in the first position at exercise is compared to determine at least one heart factor.

16. The system of claim 15, wherein the at least one heart factor comprises a pressure in the circulatory system of the patient and wherein the circulatory system comprises a vessel.

17. The system of claim 16, wherein the vessel comprises a pulmonary artery or a central venous system.

18. A system for assessment and management of congestion in heart failure, comprising:
an implantable sensor configured to insert into a circulatory system of a patient;
an external reader configured to selectively record a hemodynamic parameter taken from the implantable sensor; and
an accelerometer within the external reader, wherein the accelerometer is configured to detect a position of the patient, wherein the external reader is configured to record the hemodynamic parameter when the patient is in a first position and when the patient is in a second position, and wherein the hemodynamic parameter of the patient being in the first position and the hemodynamic parameter of the patient being in the second position is compared to determine at least one heart factor.

19. The system of claim 18, wherein the at least one heart factor comprises pulmonary artery pressure.

20. The system of claim 18, wherein the at least one hear factor comprises a measure of how quickly the circulatory system equilibrated when the patient moves from the first position to the second position.
